(19) United States
(12) Patent Application Publication
(10) Pub. No.: US 2004/0220504 A1
(43) Pub. Date: Nov. 4, 2004

(54) Knee orthosis

(76) Inventor: Jan Prahl, Rullstorf (DE)

Correspondence Address: Friedrich Kueffner, Suite 910, 317 Madison Avenue, New York, NY, 10017, US

(21) Appl. No.: 10/661,199

(22) Filed: Sep. 12, 2003

Related U.S. Application Data
(63) Continuation of application No. 10/267,183, filed on Oct. 9, 2002, now abandoned.

(30) Foreign Application Priority Data
Dec. 17, 1998 (DE) 298 22 445.3

(51) Int. Cl. A61F 5/00

(52) U.S. Cl. 602/26

(57) ABSTRACT

To create an orthosis for the knee (100) with at least one joint (10) which has two rails (12) slewably joined together at a swivel (17), whereby a stopper (14) is provided for on at least one rail (12), stopper which limits a maximal extension movement of the joint (10), which increases the patient's wearing comfort and acceptance, it is proposed that at least one of the rails (12) constituting the joint (10) has at least one slit (16), adjacent to the stopper (14), with an open side (18) and opposite slit walls (20), which is arranged in such a way that a striking of the rails (12) the one against the other at a maximal predetermined extended position moves the slit walls (20) the one towards the other by elastically deforming a material of the rail (12).

The invention refers to an orthosis for the knee with at least one joint which has two rails slewably joined together at a swivel, whereby a stopper is provided for on at least one rail, stopper which limits a maximal extension movement of the joint, according to the preamble of claim 1.

Knee orthosises serve to support a knee-joint of a patient, for example for the protection from injuries or accompanies the postoperative treatment of a knee disease or of a bone injury or of a ligament injury in the knee area. Conventional knee orthosises have the disadvantage that, when reaching a stopper in the joint limiting a maximal extension movement, an audible noise, which is felt as being unpleasant, arises.

The aim of this invention is to make available an improved orthosis for the knee of the above mentioned type which eliminates the above mentioned disadvantages and which increases the patient's wearing comfort and acceptance.

The aim is reached by a knee orthosis of the above mentioned type with the characteristics indicated in claim 1.

For this purpose, the invention provides for that at least one of the rails constituting the joint has at least one slit, adjacent to the stopper, with an open side and opposite slit walls, which is arranged in such a way that a striking of the rails the one against the other at a maximal predetermined stretched position moves the slit walls the one towards the other by elastically deforming a material of the rail.

This has the advantage that a noise development when reaching the stopper is considerably reduced so that the wearing comfort of the knee orthosis and thus the acceptance of the knee orthosis by the patient is substantially improved.

Preferable further developments of the device are specified in claims 2 and 3.

A reversible compression of the slit exclusively within a Hooke's area of an extension diagramm of the material of the rail is obtained by the fact that the length and width of the slit are dimensioned so that the slit walls striking the one against the other stop a compression of the slit, before a plastic deformation of the material of the rail takes place.

The material of the rails is for example titanium or steel.

The invention will be explained in detail below referring to the annexed drawings.

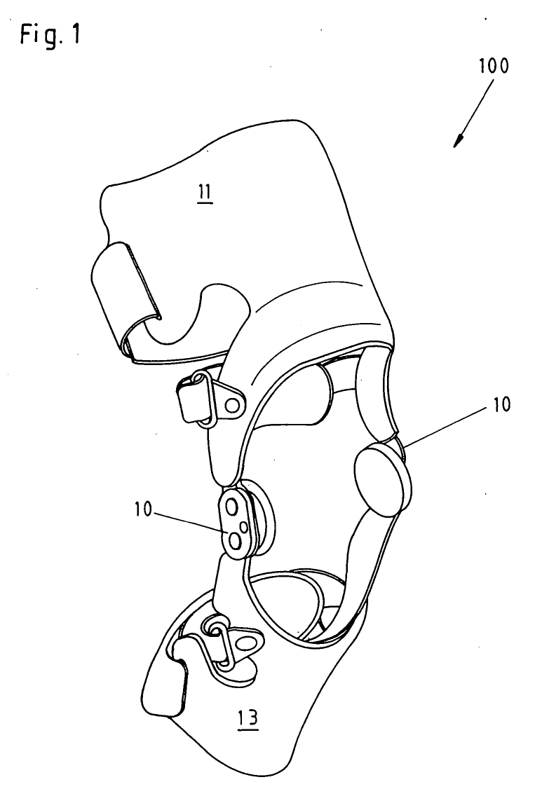

FIG. 1 shows an exploded view of a preferred embodiment of a knee orthosis according to the invention.

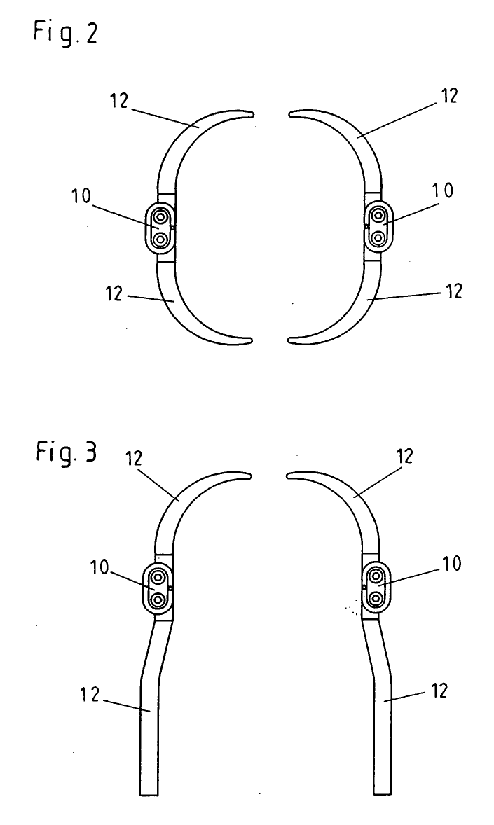

FIG. 2 shows a top view of a first preferred embodiment of a joint rail for a knee orthosis according to the invention.

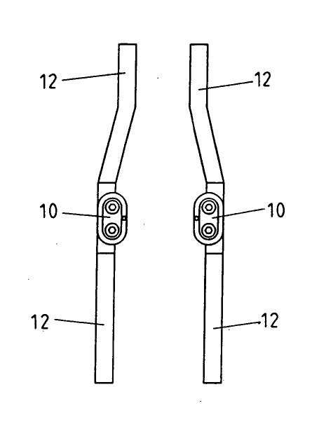

FIG. 3 shows a top view of a second preferred embodiment of a joint rail for a knee orthosis according to the invention.

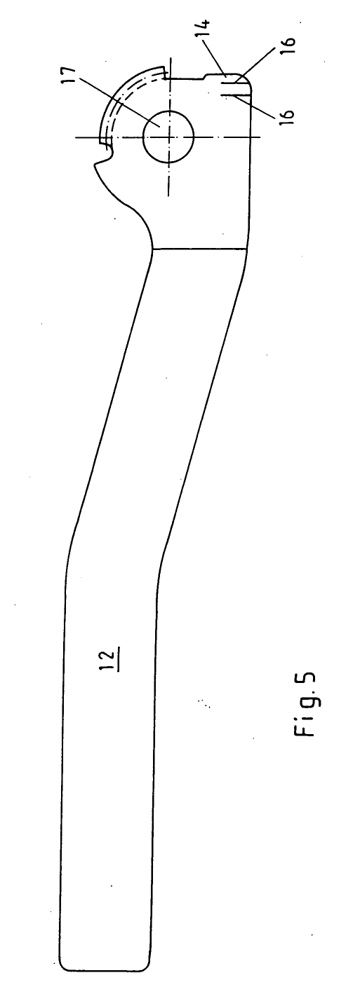

FIG. 4 shows a top view of a third preferred embodiment of a joint rail for a knee orthosis according to the invention.

FIG. 5 shows a lateral view of a preferred embodiment of a rail configured according to the invention for a knee orthosis according to the invention.

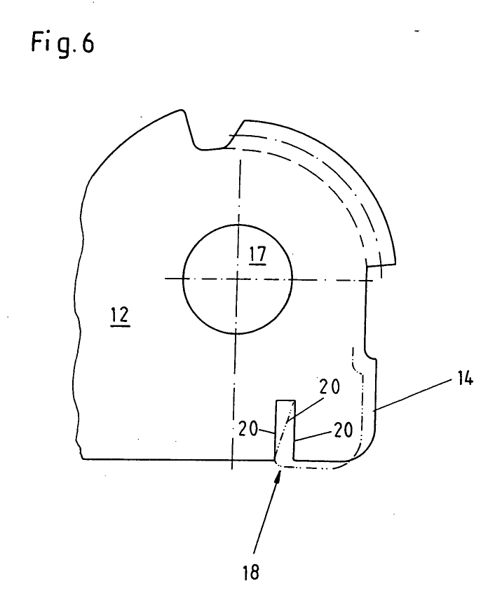

FIG. 6 shows a lateral view of an expanded detail view of a stopper of the rail of FIG. 5.

The preferred embodiment represented in FIG. 1 of a knee orthosis 100 according to the invention comprises a thigh part 11, a lower leg part 13 as well as two joints 10 which are respectively constituted by two rails, not visible in FIG. 1, and which slewably link the thigh part 11 to the lower leg part 13.

FIG. 2 to 4 illustrate different embodiments for joint constituting rails 12 for different knee orthosises. So, the knee orthosis belonging to the rails 12 according to FIG. 2 is indicated, for example, for a rupture of a front crucial ligament, for uniaxial or multiaxial instabilities of the knee-joint, for MCL injury or lateral collateral ligament injury or for early functional postoperative treatment. The knee orthosis belonging to the rails 12 according to FIG. 3 is indicated, for example, for a rupture of the posterior crucial ligament or for MCL injury or lateral collateral ligament injury. The knee orthosis belonging to the rails 12 according to FIG. 4 is indicated, for example, for gonarthrosis, instability after knee-joint replacement (total endoprosthesis) or genu recurvatum (i.e. back knee).

As may be seen in FIGS. 5 and 6, for at least one rail with a swivel 17 adjacent to a stopper 14, two slits 16 are constituted with an open side 18 and opposite slit walls 20, whereby the slits 16 are arranged in such a way that a striking of the rails 12 the one against the other at a maximal predetermined stretched position moves the slit walls 20 the one against the other under elastic deformation of a material of the rail 12. This is indicated in FIG. 6 by dotted lines. The slits 16 are, for example, built in in the stopper area with a laser and are pressed together at the stopper, which causes a noise attenuation. First tests have shown a noise attenuation of approximately 50%.

The attenuation slits 16 provided for according to the invention are narrower than a Hooke's area in the expansion diagramm. When compressing the slit-16, a straight segment of the expansion diagramm is not exceeded. The slit walls or flanks 20 of the slit 16 come to rest beforehand and cause a permanent use without wear.

1. A knee orthosis (100) with at least one joint (10) which has two rails (12) slewably joined together at a swivel (17), whereby a stopper (14) is provided for on at least one rail (12), stopper which limits a maximal extension movement of the joint (10),
characterized in
that at least one of the rails (12) constituting the joint (10) has at least one slit (16), adjacent to the stopper (14), with an open side (18) and opposite slit walls (20), which is arranged in such a way that a striking of the rails (12) the one against the other at a maximal predetermined extended position moves the slit walls (20) the one towards the other by elastically deforming a material of the rail (12).

2. A knee orthosis (100) according to claim 1,
characterized in
that the length and the width of the slit (16) are dimensioned such that the slit walls (20) striking the one against the other stop a compressing of the slit (16), before a plastic deformation of the material of the rail (12) takes place.

3. A knee orthosis (100) according to claim 1 or 2,
characterized in
that the material of the rails (12) is titanium or steel.
